(19) United States
(12) Patent Application Publication
(10) Pub. No.: US 2022/0133987 A1
(43) Pub. Date: May 5, 2022

(54) PUMP DEVICE FOR WEARABLE DRUG DELIVERY DEVICE

(71) Applicant: Insulet Corporation, Acton, MA (US)

(72) Inventor: Nicholas DENNIS, Sterling, MA (US)

(21) Appl. No.: 17/505,079

(22) Filed: Oct. 19, 2021

Related U.S. Application Data
(60) Provisional application No. 63/107,691, filed on Oct. 30, 2020.

(51) Int. Cl. A61M 5/142; A61M 5/145; A61M 5/168

(52) U.S. Cl. CPC A61M 5/14244; A61M 2205/0266; A61M 5/16881; A61M 5/145

(57) ABSTRACT

Embodiments of the present disclosure relate to techniques, processes, devices or systems for pump devices for providing a fixed volume of fluid, which is delivered and refilled within one pumping cycle. In one approach, a wearable drug delivery device may include a reservoir configured to store a liquid drug, and a drive mechanism coupled to the reservoir for receiving the liquid drug. The drive mechanism may include a housing defining a chamber, the housing including an inlet valve operable to receive the liquid drug and an outlet valve operable to expel the liquid drug from the chamber, and a resilient sealing member within the chamber. The drive mechanism may further include a shape memory wire coupled to the resilient sealing member, wherein the shape memory wire is operable to bias the resilient sealing member within the chamber.

### CROSS-REFERENCE TO RELATED APPLICATIONS

This application claims the benefit to U.S. Provisional Application No. 63/107,691, filed Oct. 30, 2020, the entire contents of which are incorporated herein by reference in their entirety.

### TECHNICAL FIELD

The disclosed embodiments generally relate to medication delivery. More particularly, the disclosed embodiments relate to techniques, processes, systems, and pump devices for providing a fixed volume of fluid, which is delivered and refilled within one pumping cycle.

### BACKGROUND

Many wearable drug delivery devices include a reservoir for storing a liquid drug. A drive mechanism is operated to expel the stored liquid drug from the reservoir for delivery to a user. Some conventional drive mechanisms use a plunger to expel the liquid drug from the reservoir. Accordingly, the drive mechanism generally has a length equal to a length of the reservoir. And when the reservoir is filled, these wearable drive mechanisms require a length of the drug delivery devices to be significantly larger, for example, about twice the length of the reservoir when the plunger has yet to traverse the length of the reservoir to expel fluid. Increasing the size of the drug delivery devices to accommodate filled reservoirs or pre-filled cartridges and corresponding drive mechanism components leads to bulky devices that are uncomfortable for the user to wear.

Accordingly, there is a need for a simplified system for accurately expelling a liquid drug from a reservoir, which also reduces drug delivery device size.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some approaches, a wearable drug delivery device may include a reservoir configured to store a liquid drug, and a delivery pump device including a drive mechanism coupled to the reservoir for receiving the liquid drug. The drive mechanism may include a housing defining a chamber, the housing including an inlet valve operable to receive the liquid drug and an outlet valve operable to expel the liquid drug from the chamber, a resilient sealing member within the chamber of the housing, and a shape memory wire coupled to the resilient sealing member, wherein the shape memory wire is operable to bias the resilient sealing member within the chamber.

In some approaches, a drive mechanism of a wearable drug delivery device may include a housing defining a chamber, the housing including an inlet valve operable to receive a liquid drug from a reservoir, and an outlet valve operable to expel the liquid drug from the chamber. The drive mechanism may further include a resilient sealing member within the chamber of the housing, and a shape memory alloy (SMA) wire coupled to the resilient sealing member, wherein the SMA wire is operable to bias the resilient sealing member within the chamber to modify an internal chamber pressure.

Furthermore, in some approaches, a method may include coupling a drive mechanism to a reservoir configured to store a liquid drug, the drive mechanism including a housing defining a chamber, and a resilient sealing member within the chamber of the housing, wherein the resilient sealing member and an interior surface of the housing define a liquid chamber. The drive mechanism may further include a shape memory alloy (SMA) wire coupled to the resilient sealing member. The method may further include activating the SMA wire to bias the resilient sealing member within the chamber between a first position and a second position.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates a schematic diagram of a drug delivery system according to embodiments of the present disclosure;

FIGS. 2A-2B illustrate perspective cross-sectional views of a drive mechanism of a delivery pump device according to embodiments of the present disclosure;

FIGS. 3A-3E illustrate side cross-sectional views of the drive mechanism at various stages of a pumping cycle according to embodiments of the present disclosure;

FIG. 4 illustrates a process flow of a method according to embodiments of the present disclosure; and

FIG. 5 illustrates a process flow of another method according to embodiments of the present disclosure.

The drawings are not necessarily to scale. The drawings are merely representations, not intended to portray specific parameters of the disclosure. The drawings are intended to depict exemplary embodiments of the disclosure, and therefore are not be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure will be thorough and complete, and will fully convey the scope of methods and devices to those skilled in the art. Each of the systems, devices, and methods disclosed herein provides one or more advantages over conventional systems, components, and methods.

Embodiments of the present disclosure provide a delivery pump device including an integral shape memory alloy (SMA) wire and resilient sealing member to draw in and expel a fixed volume of fluid (e.g., liquid drug) from a chamber. In some embodiments, the chamber includes an inlet and an outlet, each including a check valve to enable one-way flow into or out of the chamber. Upon activation/contraction of the SMA wire, the resilient sealing member is translated upward within the chamber, compressing the volume and increasing internal chamber pressure. The increased positive pressure eventually opens the outlet valve to deliver the volume of fluid to a cannula or microneedle array. The fixed volume may be a function of internal chamber geometries and SMA stroke. Once the SMA wire fully contracts, the resilient sealing member will be in a compressed state.

When the SMA wire is deactivated, the SMA wire will start to relax and the stored energy of the resilient sealing member will cause the resilient sealing member to spring back to an original position. This motion of the resilient sealing member creates a negative pressure differential in the chamber, thus causing the inlet check valve to open, and drawing fluid back into the chamber. The cycle is then repeated by activating the SMA wire. Advantageously, the delivery pump device of the present disclosure enables a fixed volume of fluid to be delivered and refilled without any secondary steps or additional components. Said another way, the system design and material properties of the SMA wire dictate the fluid response into and out of the chamber.

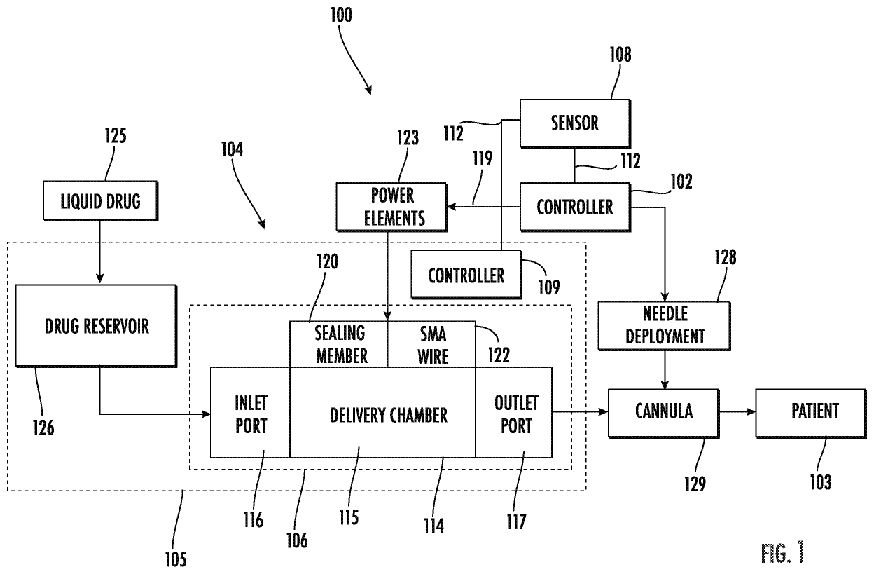

FIG. 1 illustrates a simplified block diagram of an example system (hereinafter “system”) 100. The system 100 may be a wearable or on-body drug delivery device attached to the skin of a patient 103. The system 100 may include a controller 102, a pump mechanism 104 (hereinafter “pump 104”), and a sensor 108. The sensor 108 may be a glucose or other analyte monitor such as, for example, a continuous glucose monitor. The sensor 108 may, for example, be operable to measure blood glucose (BG) values of a user to generate a measured BG level signal 112. The controller 102, the pump 104, and the sensor 108 may be communicatively coupled to one another via a wired or wireless communication path. For example, each of the controller 102, the pump 104 and the sensor 108 may be equipped with a wireless radio frequency transceiver operable to communicate via one or more communication protocols, such as Bluetooth®, or the like. As will be described in greater detail herein, the system 100 may also include a delivery pump device (hereinafter “device”) 105, which includes a drive mechanism 106 having a housing 114 defining a chamber 115, an inlet port 116, and an outlet port 117. The drive mechanism 106 may further include a resilient sealing member 120 within the chamber 115, the resilient sealing member 120 connected to a SMA wire 122. The system 100 may include additional components not shown or described for the sake of brevity.

The controller 102 may receive a desired BG level signal, which may be a first signal, indicating a desired BG level or range for the patient 103. The desired BG level signal may be stored in memory of a controller 109 on device 105, received from a user interface to the controller 102, or another device, or by an algorithm within controller 109 (or controller 102) that automatically determines a BG level for the patient 103. The sensor 108 may be coupled to the patient 103 and operable to measure an approximate value of a BG level of the user. In response to the measured BG level or value, the sensor 108 may generate a signal indicating the measured BG value. As shown in the example, the controller 102 may also receive from the sensor 108 via a communication path, the measured BG level signal 112, which may be a second signal.

Based on the desired BG level signal and the measured BG level signal 112, the controller 102 or controller 109 may generate one or more control signals for directing operation of the pump 104. For example, one control signal 119 from the controller 102 or controller 109 may cause the pump 104 to turn on, or activate one or more power elements 123 operably connected with the device 105. As will be described in greater detail herein, the power element 123 may activate the SMA wire 122, causing the SMA wire 122 to change shape and/or length, which in turn will change a configuration of the resilient sealing member 120. The specified amount of a liquid drug 125 (e.g., insulin) may then be drawn into the chamber 115, through the inlet port 116, in response to a change in pressure due to the change in configuration of the resilient sealing member 120. Ideally, the specified amount of the liquid drug 125 may be determined based on a difference between the desired BG level signal and the actual BG signal level 112. The specified amount of the liquid drug 125 may be determined as an appropriate amount of insulin to drive the measured BG level of the user to the desired BG level. Based on operation of the pump 104, as determined by the control signal 119, the patient 103 may receive the liquid drug from a reservoir 126. The system 100 may operate as a closed-loop system, an open-loop system, or as a hybrid system. In an exemplary closed-loop system, the controller 109 may direct operation of the device 105 without input from the controller 102, and may receive BG level signal 112 from the sensor 108. The sensor 108 may be housed within the device 105 or may be housed in a separate device and communicate wirelessly directly with the device 105.

As further shown, the system 100 may include a needle deployment component 128 in communication with the controller 102 or the controller 109. The needle deployment component 128 may include a needle/cannula 129 deployable into the patient 103 and may have one or more holes at a distal end thereof. The cannula 129 may form a portion of a fluid path coupling the patient 103 to the reservoir 126. More specifically, the inlet port 116 may be coupled to the reservoir 126 by a first fluid path component 130. The first fluid path component 130 may be of any size and shape and may be made from any material. The first fluid path component 130 can allow fluid, such as the liquid drug 125 in the reservoir 126, to be transferred to the device 105 through the inlet port 116.

As further shown, the outlet port 117 may be coupled to the cannula 129 by a second fluid path component 131. The second fluid path component 131 may be of any size and shape and may be made from any material. The second fluid path component 131 may be connected to the cannula 129 to allow fluid expelled from the device 105 to be provided to the patient 103. The first and second fluid path components 130 and 131 may be rigid or flexible.

The controller 102/109 may be implemented in hardware, software, or any combination thereof. The controller 102/109 may, for example, be a processor, a logic circuit or a microcontroller coupled to a memory. The controller 102/109 may maintain a date and time as well as other functions (e.g., calculations or the like) performed by processors. The controller 102/109 may be operable to execute an artificial pancreas (AP) algorithm stored in memory (not shown) that enables the controller 102/109 to direct operation of the pump 104. For example, the controller 102/109 may be operable to receive an input from the sensor 108, wherein the input indicates an automated insulin delivery (AID) application setting. Based on the AID application setting, the controller 102/109 may modify the behavior of the pump 104 and resulting amount of the liquid drug 125 to be delivered to the patient 103 via the device 105.

In some embodiments, the sensor 108 may be, for example, a continuous glucose monitor (CGM). The sensor 108 may be physically separate from the pump 104, or may be an integrated component within a same housing thereof. The sensor 108 may provide the controller 102 with data indicative of measured or detected blood glucose levels of the user.

The power element 123 may be a battery, a piezoelectric device, or the like, for supplying electrical power to the device 105. In other embodiments, the power element 123, or an additional power source (not shown), may also supply power to other components of the pump 104, such as the controller 102, memory, the sensor 108, and/or the needle deployment component 128.

In an example, the sensor 108 may be a device communicatively coupled to the controller 102 and may be operable to measure a blood glucose value at a predetermined time interval, such as approximately every 5 minutes, 10 minutes, or the like. The sensor 108 may provide a number of blood glucose measurement values to the AP application.

In some embodiments, the pump 104, when operating in a normal mode of operation, provides insulin stored in the reservoir 126 to the patient 103 based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the sensor 108 or other functional elements of the pump 104. For example, the pump 104 may contain analog and/or digital circuitry that may be implemented as the controller 102/109 for controlling the delivery of the drug or therapeutic agent. The circuitry used to implement the controller 102/109 may include discrete, specialized logic and/or components, an application-specific integrated circuit, a microcontroller or processor that executes software instructions, firmware, programming instructions or programming code enabling, for example, an AP application stored in memory, or any combination thereof. For example, the controller 102/109 may execute a control algorithm and other programming code that may make the controller 102/109 operable to cause the pump to deliver doses of the drug or therapeutic agent to a user at predetermined intervals or as needed to bring blood glucose measurement values to a target blood glucose value. The size and/or timing of the doses may be pre-programmed, for example, into the AP application by the patient 103 or by a third party (such as a health care provider, a parent or guardian, a manufacturer of the wearable drug delivery device, or the like) using a wired or wireless link.

Although not shown, in some embodiments, the sensor 108 may include a processor, memory, a sensing or measuring device, and a communication device. The memory may store an instance of an AP application as well as other programming code and be operable to store data related to the AP application.

In various embodiments, the sensing/measuring device of the sensor 108 may include one or more sensing elements, such as a blood glucose measurement element, a heart rate monitor, a blood oxygen sensor element, or the like. The sensor processor may include discrete, specialized logic and/or components, an application-specific integrated circuit, a microcontroller or processor that executes software instructions, firmware, programming instructions stored in memory, or any combination thereof.

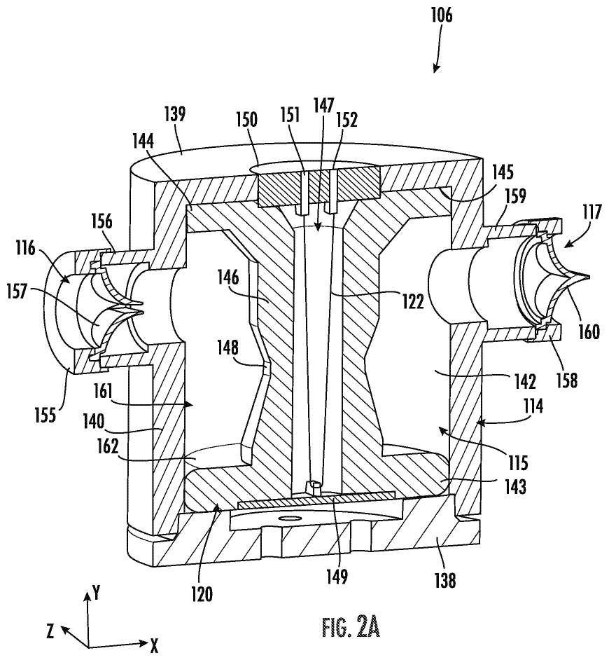

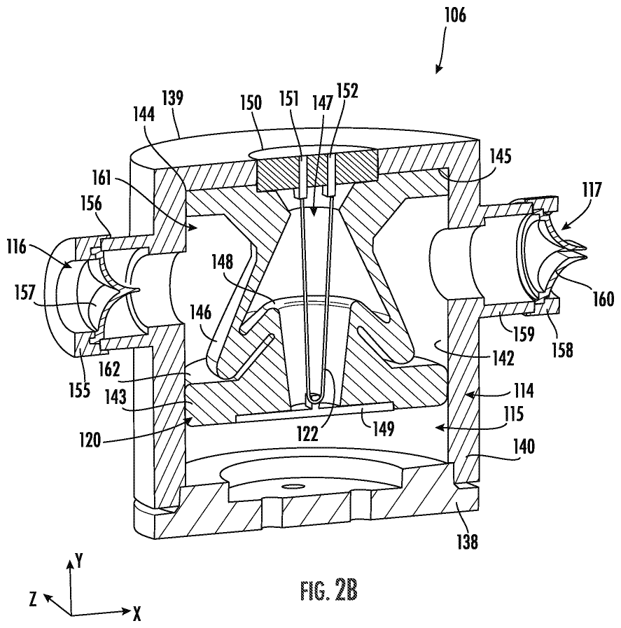

Turning now to FIGS. 2A-2B, the drive mechanism 106 according to embodiments of the present disclosure will be described in greater detail. As shown, the drive mechanism 106 may include the housing 114 defining the chamber 115. The housing 114 may include a bottom wall 138 opposite a top wall 139, and a sidewall 140 extending between the bottom wall 138 and the top wall 139. An interior surface 142 of the sidewall 140 may partially define the chamber 115. Although shown generally as cylindrically shaped, the sidewall 140, the bottom wall 138, and/or the top wall 139 may take on a different configuration in alternative embodiments.

As further shown, the drive mechanism 106 includes the resilient sealing member 120 within the chamber 115. The resilient sealing member 120 may include a first flange 143 in direct contact with the interior surface 142 of the sidewall 140 to form a seal therebetween. The resilient sealing member 120 may further include a second flange 144 fixed to an underside 145 of the top wall 139 and/or the interior surface 142 of the sidewall 140. During use, the second flange 144 is generally stationary, while the first flange 143 is permitted to move between the bottom wall 138 and the top wall 139, e.g., along the y-direction. The resilient sealing member 120 may further include a central section 146 extending between the first and second flanges 143, 144. In some embodiments, the central section 146 may have a varied thickness, e.g., along the x-direction and/or the z-direction. Specifically, the central section 146 may include one or more weakened areas 148 to promote folding or collapsing of the resilient sealing member 120 as the first flange 143 is brought towards the second flange 144. In other embodiments, the central section 146 may have a substantially constant thickness.

Although non-limiting, the resilient sealing member 120 may be made from a shape memory polymer, such as a polymeric smart material, which has the ability to return from a temporary deformed/compressed shape to a permanent shape. For example, the configuration of the resilient sealing member 120 in FIG. 2A may correspond to its natural or permanent shape, while the configuration of the resilient sealing member 120 in FIG. 2B may correspond to the temporary deformed/compressed shape.

The SMA wire 122 of the drive mechanism 106 may extend through a channel 147 of the resilient sealing member 120. As shown, the SMA wire 122 may be connected with a base plate 149, which may be in contact (e.g., beneath) the resilient sealing member 120 to bias the resilient sealing member 120 between a first position, such as the position demonstrated in FIG. 2A, and a second position, such as the position demonstrated in FIG. 2B. The base plate 149 may provide support and rigidity to the first flange 143 of the resilient sealing member 120. In some embodiments, the base plate 149 is fixed to the first flange 143.

The SMA wire 122 can be coupled to the power element 123 (FIG. 1) by way of a contact 150, a first pole or connector 151, and a second pole or connector 152. The power element 123 can be used to energize both legs/sides of the SMA wire 122, as further described herein. The first connector 151 may be coupled to a first output of the power element 132 (e.g., a positive output terminal), and the second connector 152 can be coupled to a second output of the power element 132 (e.g., a negative output terminal). The contact 150 can be connected to ground or a ground terminal.

During use, the power element 132 may be activated to energize the SMA wire 122, which causes the SMA wire 122 to change shape (e.g., contract). More specifically, the activated SMA wire 122 begins to shorten (e.g., along the y-direction), after having previously been passively relaxed, pulling the base plate 149 and the first flange 143 towards the top wall 139 of the housing 114, as the SMA wire 122 strives to return to its memorized or natural/pre-stressed shape and length. In various embodiments, contraction of the SMA wire 122 may be controlled by increasing or decreasing heat generated by the power element 132. For example, a lower current supplied to the SMA wire 122 may cause the base plate 149 to move more slowly than a higher current.

Although non-limiting, the SMA wire 122 may generally be V-shaped or U-shaped, with a base of the SMA wire (i.e., the area where both legs meet) coupled to the base plate 149. As a result, the total force exerted by each leg may be summed, with the total pulling force accordingly doubled for a U or V-shaped arrangement. For a given required total force, thinner SMA wires may accordingly be used, with the electric resistance increasing with decreasing diameter. Further, since the electric resistance depends on the total length of the SMA wire 122, the electric resistances of a U-shaped arrangement is accordingly double the electric resistance of a single leg of identical diameter. The double leg configuration of the SMA wire 122 accordingly results in a comparatively high electric resistance, which is favorable in order to limit the required current for heating. In other embodiments, alternative folding arrangements are possible, such as a threefold (resulting in an “N-shape”) or a fourfold (resulting in an “M-shape”).

The drive mechanism 106 may further include the inlet port 116 and the outlet port 117. As shown, the inlet port 116 may include an inlet cap 155 coupled to an inlet cylinder 156. An inlet valve 157, such as a check valve or one-way valve, may be positioned within the inlet cylinder 156. Similarly, the outlet port 117 may include an outlet cap 158 coupled to an outlet cylinder 159. An outlet valve 160, which may also be a check valve or one-way valve, is positioned within the outlet cylinder 159. The inlet valve 157 is configured to permit the liquid drug 125 to only flow into the chamber 115, while the outlet valve 160 is configured to permit the liquid drug 125 to only flow out of the chamber 115.

When the inlet valve 157 is opened, as shown in FIG. 2A, the liquid drug 125 flows through the inlet cylinder 156 and into a liquid chamber 161, which may be an area of the chamber 115 defined by an outer surface 162 of the resilient sealing member 120 and the interior surface 142 of the housing 114. A volume of the liquid chamber 161 may change as the resilient sealing member 120 changes configuration. For example, as the resilient sealing member 120 moves towards the top wall 139 of the housing 114, the volume of the liquid chamber 161 decreases, which increases pressure within the housing, causing the outlet valve 160 to open, as shown in FIG. 2B. In various embodiments, a length (e.g., along the x-direction) of the inlet cylinder 156 and the outlet cylinder 159 may be the same or different. Furthermore, an inner diameter of the inlet cylinder 156 and the outlet cylinder 159 may be the same or different.

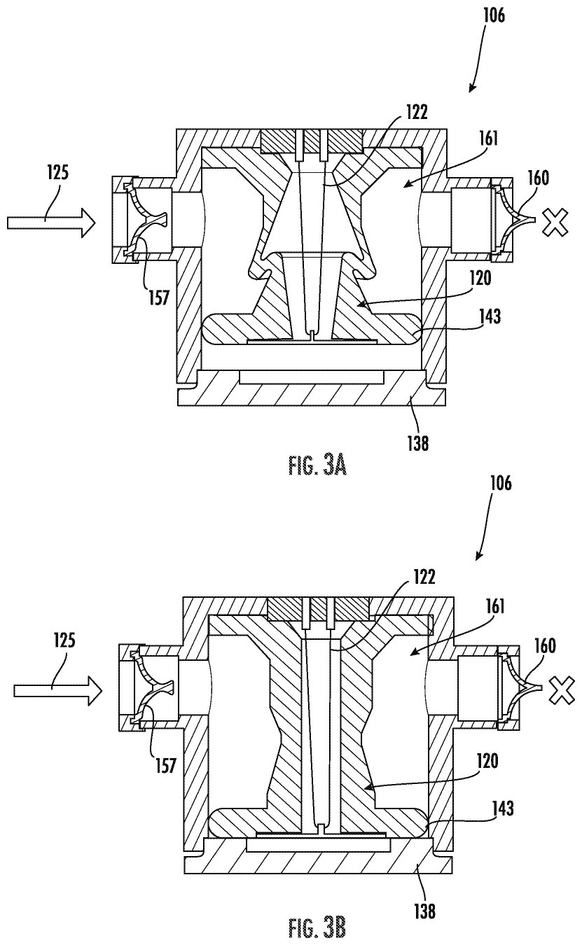

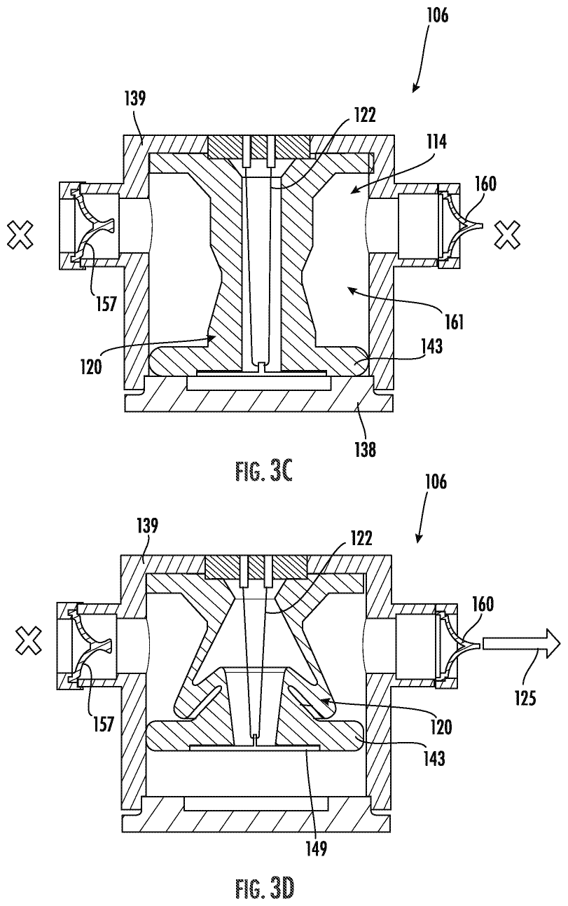

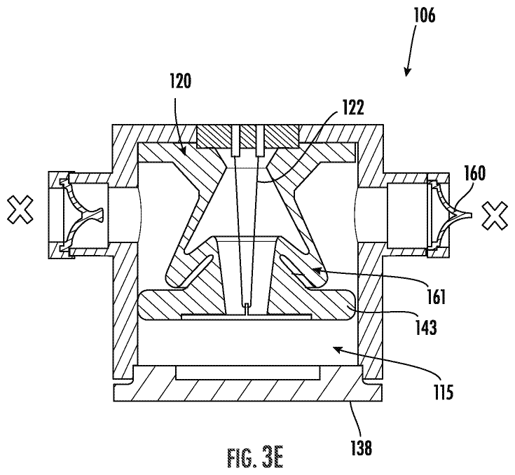

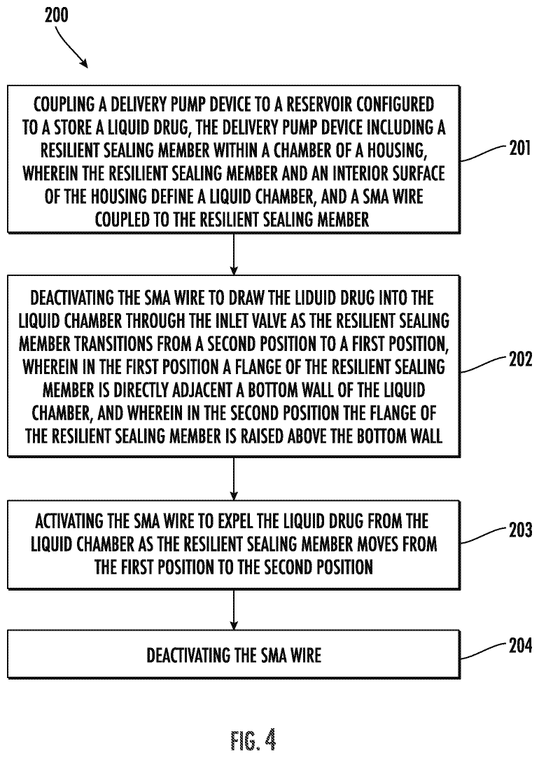

FIGS. 3A-3E illustrate the drive mechanism 106 at various stages of a filling and pumping cycle according to embodiments of the present disclosure. Although not demonstrated, the drive mechanism 106 may first be primed to fill the liquid chamber 161, e.g., by repeatedly cycling in fluid and expelling air from the inlet port 116, the liquid chamber 161, and the outlet port 117. In some embodiments, the outlet cylinder 159 may be pointing upward during the priming such that the outlet cylinder 159 is at the top of the drive mechanism 106, allowing air to exit as fluid fills the liquid chamber 161. In FIG. 3A, the SMA wire 122 and the resilient sealing member 120 are in an intermediate contracted position in which the first flange 143 is raised above the bottom wall 138. At this stage, the SMA wire 122 may have been recently deactivated, thus causing the SMA wire 122 to begin relaxing towards the bottom wall 138 as it cools. As the first flange 143 descends towards the bottom wall 138, pressure in the liquid chamber 161 decreases, causing the outlet valve 160 to remain closed and the inlet valve 157 to open to permit the liquid drug 125, or additional liquid drug 125, to enter the liquid chamber 161.

As demonstrated in FIG. 3B, the SMA wire 122 has now cooled and relaxed, and the resilient sealing member 120 has returned to an expanded, original position in which the first flange 143 rests on, or is positioned directly adjacent, the bottom wall 138. Negative pressure created in the liquid chamber 161 by the reconfiguration of the resilient sealing member 120 causes the liquid drug 125 to flow through the inlet valve 157 and into the liquid chamber 161. The outlet valve 160 remains closed.

As the liquid drug 125 stops filling the liquid chamber 161, the drive mechanism 106 may enter a neutral state, which is demonstrated in FIG. 3C. As a pressure differential between the inside and outside of the liquid chamber 161 becomes balanced (due to liquid drug 125 entering the liquid chamber 161), the liquid drug 125 may no longer be drawn into the liquid chamber 161 through the inlet valve 157. The SMA wire 122 is unactivated, and the first flange 143 of the resilient sealing member 120 remains along the bottom wall 138 of the housing 114. The inlet valve 157 and the outlet valve 160 are closed.

As demonstrated in FIG. 3D, the SMA wire 122 is then activated, e.g., in response to a current received from the power element 132. The activated SMA wire 122 contracts towards the top wall 139 of the housing 114, causing the base plate 149 and first flange 143 to rise to a fully contracted position and the resilient sealing member 120 to deform. As shown, the inlet valve 157 is closed, and the increased pressure in the liquid chamber 161, due to the decreasing spatial volume, causes the outlet valve 160 to open. The liquid drug 125 may then be expelled through the outlet valve 160 and delivered to the second fluid path component 131 (FIG. 1).

In FIG. 3E, current to the SMA wire 122 may be decreased or discontinued, causing the first flange 143 to stop rising within the chamber 115 and the liquid drug 125 to stop exiting the liquid chamber 161 through the outlet valve 160. The inlet valve 157 and the outlet valve 160 may be closed. As the SMA wire 122 further relaxes, the first flange 143 descends towards the bottom wall 138, e.g., to the position shown in FIG. 3A, due to the stored energy and spring-like force of the resilient sealing member 120. The pressure within the liquid chamber 161 may then decrease to again draw the liquid drug 125 through the inlet valve 157. In some embodiments, deactivation of the SMA wire 122, e.g., by the controller 102, may occur following expiration of a predetermined time period triggered by the initial activation of the SMA wire 122. The predetermined time period may be sufficient to allow a controlled dose (e.g., 0.05 mL or 0.025 mL) of the liquid drug 125 to enter and then be extinguished from the liquid chamber 161. In other words, the difference in volume of the liquid chamber 161 between the first position, when a bottom surface of the flange 143 of the resilient sealing member 120 is directly adjacent the bottom wall 138 of the liquid chamber 161 (as shown, for example, in FIG. 2A) and the second position, when the flange 143 is in an elevated position (as shown, for example, in FIG. 2B), can be equal to one dose of liquid drug to be delivered out of outlet valve 160 (or outlet port 117) by drive mechanism 106, to cannula 129 and ultimately to patient 103. One dose of liquid drug may be equal to, for example, 0.05 mL or 0.025 mL or less than 1 mL.

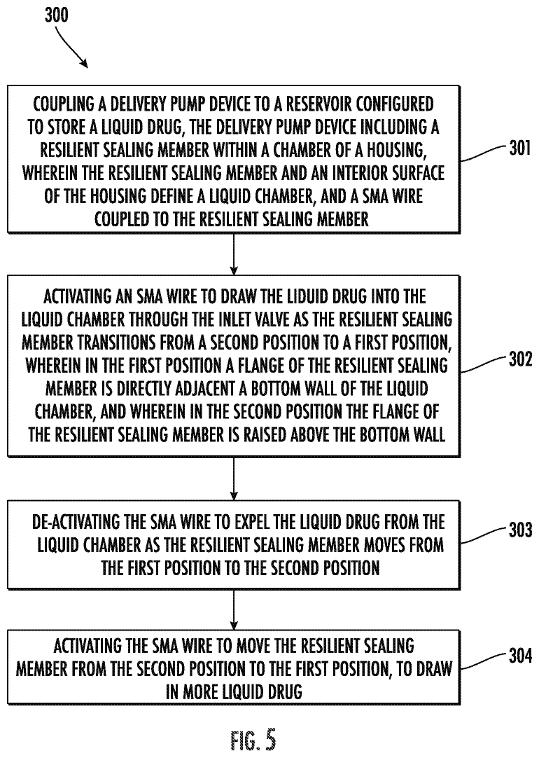

FIG. 4 illustrates an example process 200 according to embodiments of the present disclosure. At block 201, the process 200 may include coupling a drive mechanism of a delivery pump device to a reservoir configured to store a liquid drug, the drive mechanism including a resilient sealing member within a chamber of a housing, wherein the resilient sealing member and an interior surface of the housing define a liquid chamber, and a SMA wire coupled to the resilient sealing member. In some embodiments, a seal is formed between a flange of the resilient sealing member and an interior surface of the chamber of the housing. In some embodiments, the drive mechanism may include an inlet valve along one side of the housing, and an outlet valve along another side of the housing. The inlet valve may be a check valve positioned within an inlet port, while the outlet valve may be a check valve positioned within an outlet port. The inlet and outlet ports may be in fluid communication with the liquid chamber.

At block 202, the process 200 may include deactivating the SMA wire to draw the liquid drug into the liquid chamber through the inlet valve as the resilient sealing member transitions from a second position to a first position, wherein in the first position a flange of the resilient sealing member is directly adjacent a bottom wall of the liquid chamber, and wherein in the second position the flange of the resilient sealing member is raised above the bottom wall. In some embodiments, the resilient sealing member may be deformed or compressed in the second position and expanded or relaxed in the first position.

The drive mechanism may then enter a neutral state in which a pressure differential between the inside and outside of the liquid chamber becomes balanced, e.g., due to the liquid drug entering the liquid chamber 161. As a result, the liquid drug may no longer be drawn into the liquid chamber through the inlet valve. The SMA wire is unactivated, and the flange of the resilient sealing member is positioned along the bottom wall of the housing. The inlet valve and the outlet valve are closed.

At block 203, the process 200 may include activating the SMA wire to expel the liquid drug from the liquid chamber as the resilient sealing member moves from the first position to the second position. The outlet valve, which may be a check valve, opens in response to increased pressure within the liquid chamber from the decreased spatial volume of the liquid chamber caused by movement of the resilient sealing member from the first position to the second position.

At block 204, the process 200 may include deactivating the SMA wire, which causes the flange of the resilient sealing member to stop rising and the liquid drug to stop exiting the liquid chamber through the outlet valve. The SMA wire may then start to relax, causing the resilient sealing member to expand and return to its natural or permanent shape. In some embodiments, deactivation of the SMA wire, e.g., by a controller, may occur following expiration of a predetermined time period triggered by the initial activation of the SMA wire. The predetermined time period may be sufficient to allow a controlled dose (e.g., 0.05 mL or 0.025 mL) of the liquid drug to enter and then be extinguished from the liquid chamber.

FIG. 5 illustrates an example process 300 according to an alternative embodiment of the present disclosure in which an SMA wire, a motor, or other mechanism moves the resilient sealing member from a relaxed elevated position to an expanded or stressed/extended position. In this alternative embodiment, the SMA wire may be positioned on an opposite or bottom wall 138 as compared with, for example in FIG. 2B (where the SMA wire is positioned at a top wall 139). At block 301, the process 300 may include coupling a drive mechanism of a delivery pump device to a reservoir configured to store a liquid drug, the drive mechanism including a resilient sealing member within a chamber of a housing, wherein the resilient sealing member and an interior surface of the housing define a liquid chamber, and a SMA wire coupled to the resilient sealing member. In some embodiments, a seal is formed between a flange of the resilient sealing member and an interior surface of the chamber of the housing. In some embodiments, the drive mechanism may include an inlet valve along one side of the housing, and an outlet valve along another side of the housing. The inlet valve may be a check valve positioned within an inlet port, while the outlet valve may be a check valve positioned within an outlet port. The inlet and outlet ports may be in fluid communication with the liquid chamber.

At block 302, the process 300 may include activating an SMA wire to draw the liquid drug into the liquid chamber through the inlet valve as the resilient sealing member transitions from a second position to a first position, wherein in the first position a flange of the resilient sealing member is directly adjacent a bottom wall of the liquid chamber, and wherein in the second position the flange of the resilient sealing member is raised above the bottom wall. In this alternative embodiment, the resilient sealing member may be relaxed in the second (elevated) position, and deformed or expanded in the first position (such that the flange is adjacent the bottom wall of liquid chamber); and the SMA wire may move the resilient sealing member from the relaxed second position to the expanded first position when energized; and upon the SMA wire being de-energized, the resilient sealing member may relax and the stored energy of the resilient sealing member will cause the resilient sealing member to spring back to an original position (i.e., an elevated or second position in this embodiment).

When the flange is directly adjacent or touching the bottom wall of the liquid chamber, the system may be in a neutral pressure state in which a pressure differential between the inside and outside of the liquid chamber becomes balanced, e.g., due to the liquid drug entering the liquid chamber. As a result, the liquid drug may no longer be drawn into the liquid chamber through the inlet valve.

At block 303, the process 300 may include de-activating the SMA wire to expel the liquid drug from the liquid chamber as the resilient sealing member moves from the first position to the second position. The outlet valve, which may be a check valve, opens in response to increased pressure within the liquid chamber from the decreased spatial volume of the liquid chamber caused by movement of the resilient sealing member from the first position (stressed) to the second position (unstressed).

At block 304, the flange of the resilient sealing member stops rising and the liquid drug stops exiting the liquid chamber through the outlet valve. The SMA wire may then be activated again to move the resilient sealing member from the second position to the first position, to draw in more liquid drug, thus repeating the cycle. In some embodiments, deactivation of the SMA wire, e.g., by a controller, may occur following expiration of a predetermined time period triggered by the initial activation of the SMA wire. The predetermined time period may be sufficient to allow a controlled dose (e.g., 0.05 mL or 0.025 mL) of the liquid drug to enter and then be extinguished from the liquid chamber. Alternatively, deactivation may be triggered when the resilient sealing member reaches a particular position within the fluid chamber. Electrodes may be placed within flange the and in an outer wall of the liquid chamber and charged such that when the electrodes come in contact with each other, the controller can take a certain action, such as de-activation of the SMA wire.

As used herein, the algorithms or computer applications that manage blood glucose levels and insulin therapy may be referred to as an “artificial pancreas” algorithm-based system, or more generally, an artificial pancreas (AP) application. An AP application may be programming code stored in a memory device and that is executable by a processor, controller or computer device.

The techniques described herein for a drug delivery system (e.g., the system 100 or any components thereof) may be implemented in hardware, software, or any combination thereof. Any component as described herein may be implemented in hardware, software, or any combination thereof. For example, the system 100 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed devices may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A wearable drug delivery device, comprising:
a reservoir configured to store a liquid drug;
a delivery pump device including a drive mechanism coupled to the reservoir for receiving the liquid drug, the drive mechanism comprising:
a housing defining a chamber, the housing including an inlet valve operable to receive the liquid drug and an outlet valve operable to expel the liquid drug from the chamber;
a resilient sealing member within the chamber of the housing; and
a shape memory wire coupled to the resilient sealing member, wherein the shape memory wire is operable to bias the resilient sealing member within the chamber.

2. The wearable drug delivery device of claim 1, the drive mechanism further comprising a base plate coupled to the shape memory wire, wherein the base plate is in contact with the resilient sealing member to bias the resilient sealing member between a first position and a second position.

3. The wearable drug delivery device of claim 1, the resilient sealing member comprising a flange in direct physical contact with an interior surface defining the chamber of the housing.

4. The wearable drug delivery device of claim 1, the shape memory wire extending through a channel of the resilient sealing member.

5. The wearable drug delivery device of claim 1, further comprising:
a first fluid path component connecting the reservoir with an inlet port of the housing; and
a second fluid path component connecting an outlet port of the housing with a cannula, wherein the inlet valve is positioned within the inlet port, and wherein the outlet valve is positioned within the outlet port.

6. The wearable drug delivery device of claim 1, wherein at least one of the inlet and outlet valves is a check valve.

7. The wearable drug delivery device of claim 1, wherein the resilient sealing member is directly connected to a top wall of the housing.

8. The wearable drug delivery device of claim 1, further comprising a power source coupled to the shape memory wire, wherein power from the power source causes the shape memory wire to contract.

9. The wearable drug delivery device of claim 8, further comprising a controller communicatively coupled to the power source, wherein the controller is operable to:
receive an input indicating an automated insulin delivery (AID) application setting; and
in response to the input, activate the power source.

10. A drive mechanism of a wearable drug delivery device, the drive mechanism comprising:
a housing defining a chamber, the housing including an inlet valve operable to receive a liquid drug from a reservoir, and an outlet valve operable to expel the liquid drug from the chamber;
a resilient sealing member within the chamber of the housing; and
a shape memory alloy (SMA) wire coupled to the resilient sealing member, wherein the SMA wire is operable to bias the resilient sealing member within the chamber to modify an internal chamber pressure.

11. The drive mechanism of claim 10, further comprising a base plate coupled to the SMA wire, wherein the base plate is coupled to the resilient sealing member to bias the resilient sealing member between a first position and a second position.

12. The drive mechanism of claim 10, the resilient sealing member comprising:
a first flange in direct physical contact with an interior surface defining the chamber of the housing; and
a second flange directly coupled to a top wall of the housing.

13. The drive mechanism of claim 10, the SMA wire extending through a channel of the resilient sealing member.

14. The drive mechanism of claim 10, further comprising:
a first fluid path component connecting the reservoir with an inlet port of the housing; and
a second fluid path component connecting an outlet port of the housing with a cannula, wherein the inlet valve is positioned within the inlet port, and wherein the outlet valve is positioned within the outlet port.

15. A method, comprising:
coupling a drive mechanism to a reservoir configured to store a liquid drug, the drive mechanism comprising:
a housing defining a chamber;
a resilient sealing member within the chamber of the housing, wherein the resilient sealing member and an interior surface of the housing define a liquid chamber; and
a shape memory alloy (SMA) wire coupled to the resilient sealing member; and
activating the SMA wire to bias the resilient sealing member within the chamber between a first position and a second position.

16. The method of claim 15, further comprising:
providing an inlet valve along one side of the housing;
providing an outlet valve along another side of the housing; and
deactivating the SMA wire to draw the liquid drug into the liquid chamber through the inlet valve as the resilient sealing member transitions from the second position to the first position, wherein in the first position a flange of the resilient sealing member is directly adjacent a bottom wall of the liquid chamber, and wherein in the second position the flange of the resilient sealing member is raised above the bottom wall.

17. The method of claim 16, further comprising expelling the liquid drug from the liquid chamber by moving the resilient sealing member from the first position to the second position, wherein the outlet valve opens in response to increased pressure within the liquid chamber as the resilient sealing member moves from the first position to the second position.

18. The method of claim 17, further comprising opening the inlet valve in response to decreased pressure within the liquid chamber caused by movement of the resilient sealing member from the second position to the first position, wherein the inlet valve and the outlet valve are each one-way check valves.

19. The method of claim 16, further comprising forming a seal between the flange of the resilient sealing member and the interior surface of the chamber of the housing.

20. The method of claim 15, further comprising coupling the resilient sealing member to a top wall of the housing.
